(19) United States
(12) Patent Application Publication
(10) Pub. No.: US 2011/0046058 A1
(43) Pub. Date: Feb. 24, 2011

(54) COMPOSITIONS FOR ENTERAL ABSORPTION AND SUSTAINED ACTION OF LEPTIN-RELATED PEPTIDES USEFUL IN THE TREATMENT OF OBESITY AND LEPTIN-MODULATED DISEASE

(75) Inventors: Edward T. Maggio, San Diego, CA (US); Patricia Grasso, Newtown, CT (US)

Correspondence Address: DLA PIPER LLP (US), 4365 EXECUTIVE DRIVE, SUITE 1100, SAN DIEGO, CA, 92121-2133, US

(73) Assignees: AEGIS THERAPEUTICS LLC, San Diego, CA (US); ALBANY MEDICAL COLLEGE, Albany, NY (US)

(21) Appl. No.: 12/862,626

(22) Filed: Aug. 24, 2010

Related U.S. Application Data
(60) Provisional application No. 61/236,396, filed on Aug. 24, 2009.

(51) Int. Cl. A61K 38/22

(52) U.S. Cl. 514/9.7

(57) ABSTRACT

The invention relates generally to enterally absorbed peptide compositions and more specifically to leptin-related peptide compositions, methods of administration, and methods of modulating the speed and sustained action of systemic absorption of such peptides useful in the treatment of obesity and other leptin modulated diseases.

### CROSS REFERENCE TO RELATED APPLICATION(S)

This application claims the benefit of priority under 35 U.S.C. §119(e) of U.S. Ser. No. 61/236,396, filed Aug. 24, 2009, the entire content of which is incorporated herein by reference.

### BACKGROUND OF THE INVENTION

1. Field of the Invention

The invention relates generally to enterally absorbed peptide compositions and more specifically to leptin peptide compositions and methods of modulating the speed and sustained action of systemic absorption of such peptides in the treatment of obesity and other leptin modulated diseases.

2. Background Information

Peptide and protein drugs are among the most useful and effective drugs yet discovered. In total, more than 140 peptide and protein drugs are currently in use today and the chemical and biological diversity available through peptides is breathtaking. Many peptides demonstrate high potency and high selectivity while exhibiting essentially no chemical toxicity. Because they are metabolized to naturally occurring amino acids, peptides and proteins do not invoke xenobiotic metabolic processes—the source of small molecule drug toxicity.

Many naturally occurring peptides have direct therapeutic applications, e.g., insulin, interferon, EPO (erythropoietin), growth hormone, and parathyroid hormone, among others. Other peptides may provide the initial biological activity from which new peptide and protein therapeutics may be designed. Important examples include a growing number of GLP-1 related peptides such as exendin-4, PYY related peptides, or leptin peptides, which promise to provide new classes of highly effective treatments for Type II diabetes and obesity.

In spite of the many attractive aspects of peptides and proteins as potential therapeutic agents, their susceptibility to denaturation, hydrolysis, and poor absorption in the gastrointestinal tract makes them unsuitable for oral administration, typically requiring administration by injection. This remains a major shortcoming.

Leptin, a protein having molecular weight of approximately 16,000 Daltons, is well-known to be a modulator of food intake. Thus, it has been studied extensively as a potential anti-obesity drug. Like most proteins, leptin is not absorbed in the gastrointestinal tract, but rather is hydrolyzed to amino acids resulting in the destruction of its activity. As a result, all previous studies conducted with leptin have required administration by injection. Injection has been carried out by subcutaneous, intraperitoneal, intramuscular, or intravenous routes. Grasso et al. discovered that certain small leptin peptide fragments preserve the anti-obesity activity of the full-length leptin molecule while providing substantial benefits over the full-length leptin molecule. These findings are described in U.S. Pat. Nos. 6,777,388, 7,186,694, 7,208,572, Australian Patent No. 772278, Grasso et al. (1997), and Novakovic et al. (2009). Such leptin peptide fragments are shown in Table 1.

TABLE 1
AMINO ACID SEQUENCES OF LEPTIN PEPTIDES

| | |
|---|---|
| (i) | S C S L P Q T; |
| | (SEQ ID NO: 1) |
| (ii) | A V P I Q K V Q D D T K T L I; |
| | (SEQ ID NO: 2) |
| (iii) | T K T L I K T I V T R I N D I; |
| | (SEQ ID NO: 3) |
| (iv) | R I N D I S H T Q S V S A K Q; |
| | (SEQ ID NO: 4) |
| (v) | V S A K Q R V T G L D F I P G; |
| | (SEQ ID NO: 5) |
| (vi) | D F I P G L H P I L S L S K M; |
| | (SEQ ID NO: 6) |
| (vii) | S L S K M D Q T L A V Y Q Q V; |
| | (SEQ ID NO: 7) |
| (viii) | V Y Q Q V L T S L P S Q N V L; |
| | (SEQ ID NO: 8) |
| (ix) | S Q N V L Q I A N D L E N L R; |
| | (SEQ ID NO: 9) |
| (x) | D L L H L L A F S K S C S L P; |
| | (SEQ ID NO: 10) |
| (xi) | S C S L P Q T S G L Q K P E S; |
| | (SEQ ID NO: 11) |
| (xii) | Q K P E S L D G V L E A S L Y; |
| | (SEQ ID NO: 12) |
| (xiii) | E A S L Y S T E V V A L S R L; |
| | (SEQ ID NO: 13) |
| (xiv) | A L S R L Q G S L Q D I L Q Q |
| | (SEQ ID NO: 14) |
| (xv) | D I L Q Q L D V S P E C; |
| | (SEQ ID NO: 15) |
| and | |
| (xvi) | S C H L P W A. |
| | (SEQ ID NO: 16) |

Some advantages of using leptin peptide fragments include lower cost of manufacture and the ability to administer these peptides noninvasively. Leptin peptide fragments that exhibit exemplary activity include the leptin peptides having the SEQ ID NO: 1 and SEQ ID NO: 16 as set forth in Table 1 and in particular their [D-Leu] analogs. The [D-Leu] analog of SEQ ID NO: 1 is also referred to as [D-Leu-4]OB3.

### SUMMARY OF THE INVENTION

It has now surprisingly been discovered that certain leptin peptides when formulated in combination with certain alkylsaccharides as described herein are stable or resistant to hydrolysis in the gastrointestinal tract and are absorbed into systemic circulation with relatively high bioavailability and a Tmax of approximately 60 minutes. These compositions may be presented for enteral absorption by ingestion using any methods known in the art, such as oral formulations, for example, tablets, capsules, liquids, rapidly disintegrating tablets or films, and the like. Alternatively, they may be administered intranasally by instillation or nasal spray. This latter route results in enteral absorption as drug is transported to the back of the throat by the normal mucociliary clearance processes, subsequently swallowed, and absorbed in the GI tract.

Accordingly, in one aspect, the present invention provides a composition including leptin peptides admixed with alkylsaccharides that provide high enteral absorption efficiency. The composition includes at least one leptin peptide; and at least one alkylsaccharide. The alkylsaccharide provides increased enteral absorption of the leptin peptide. Additionally, the composition exhibits a biphasic absorption profile when administered nasally. In various aspects, the leptin peptide of the composition includes SEQ ID NOS: 1-16. In exemplary aspects the leptin peptide has the amino acid sequence of SEQ ID NO: 1 or 16.

In various aspects, the composition exhibits specific absorption profiles. For example, the compositions may include biphasic absorption including a first Tmax of about 4 to 20 minutes and a second Tmax of about 30 to 120 minutes.

Also, the alkylsaccharide of the composition may include an alkylsaccharide having an alkyl chain including about 10 to 16 carbons. In exemplary aspects, the alkylsaccharide is n-dodecyl-beta-D-maltoside, n-tridecyl-beta-D-maltoside, n-tetradecyl beta-D-maltoside, sucrose mono-dodecanoate, sucrose mono-tridecanoate, and sucrose mono-tetradecanoate. In various aspects, the concentration of alkylsaccharide is between about 0.05% and 5% or between about 0.05% and 0.5%.

In various aspects, the composition may be formulated with a matrix suitable for buccal or sublingual delivery. Buccal or sublingual delivery can provide a means for rapid transmucosal absorption of a portion of the peptide in the presence of an absorption enhancing alkylsaccharide as described herein. Unabsorbed peptide is then swallowed and absorbed gastrointestinally, resulting in a biphasic pharmacokinetic profile providing rapid onset and sustained action. By increasing the amount of alkylsaccharide present in the formulation, the relative proportion of transmucosally or gastrointestinally absorbed peptide can be modulated. Examples of the matrices that may be used include a dissolvable thin film, lyophilized wafer which may include a bulking agent such as a protein or a saccharide, or an orally palatable liquid in the form of a syrup or spray.

In related aspects, the composition may include a mucosal delivery-enhancing agent selected from:
(a) an aggregation inhibitory agent;
(b) a charge-modifying agent;
(c) a pH control agent;
(d) a degradative enzyme inhibitory agent;
(e) a protease inhibitor such as E-64 protease inhibitor, aprotinin, leupeptin, pepstatin A, bestatin, and 4-(2-aminoethyl)benzenesulfonyl fluoride hydrochloride (AEBSF).
(f) a mucolytic or mucus clearing agent;
(g) a ciliostatic agent;
(h) a membrane penetration-enhancing agent selected from:
   a surfactant; (ii) a bile salt; (ii) a phospholipid additive, mixed micelle, liposome, or carrier; (iii) an alcohol; (iv) an enamine; (v) an NO donor compound; (vi) a long-chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x);
(i) a modulatory agent of epithelial junction physiology;
(j) a vasodilator agent;
(k) a selective transport-enhancing agent; and
(l) a stabilizing delivery vehicle, carrier, mucoadhesive, support or complex-forming species with which the compound is effectively combined, associated, contained, encapsulated or bound resulting in stabilization of the compound for enhanced nasal mucosal delivery, wherein the formulation of the compound with the intranasal delivery-enhancing agents provides for increased bioavailability of the compound in a blood plasma of a subject.

In related aspects, the invention provides a method of increasing enteral absorption and systemic circulation of a leptin peptide in a monophasic or biphasic manner comprising orally or nasally administering to a subject a composition of the present invention. In various aspects, Cₘₐₓ is increased greater than 2-fold as compared to delivery without alkylsaccharide.

In another aspect, the invention provides a method of rapidly increasing leptin peptide systemic serum concentration and providing a sustained increase over an extended period of time of leptin peptide systemic serum concentration comprising nasally administering to a subject a composition of the present invention.

In another aspect, the invention provides a method of increasing or decreasing the relative proportion of pregastric absorption of a leptin peptide as compared to gastric absorption. The method includes providing a composition of the present invention having increased or decreased concentration of alkylsaccharide, and nasally administering the composition to a subject, wherein increasing the concentration of alkylsaccharide increases the relative proportion of pregastric absorption and decreasing the concentration of alkylsaccharide decreases the relative proportion of pregastric absorption.

In another aspect, the present invention provides a composition having at least one alkylsaccharide that when admixed, mixed or blended with a leptin peptide increases the oral or enteral bioavailability of the drug.

In another aspect, the present invention provides a method of treating obesity or other leptin-related disease conditions including administering to a subject in need thereof via the oral or nasal routes a composition of the present invention.

In another aspect, the invention provides a method of increasing absorption of a leptin peptide into the CSF or brain of a subject including administering intranasally a composition of the present invention.

In another aspect, the invention provides a method of controlling caloric intake by administering a composition of the present invention.

### BRIEF DESCRIPTION OF THE DRAWINGS

FIG. 1 is a graph showing serum concentrations of mouse [D-Leu-4]OB3 5, 10, 20, 40, 60, and 120 min after oral gavage delivery of 1 mg of peptide to male Swiss Webster mice (n=6 mice per time point) in the presence and absence of dodecylmaltoside.

FIG. 2 is a graph showing serum concentrations of mouse [D-Leu-4]OB3 5, 10, 20, 40, 60, and 120 min after intraperitoneal delivery of 1 mg of peptide to male Swiss Webster mice (n=6 mice per time point). Each value represents mean±SEM. Error bars are contained within the point and ranged between 0.01 and 0.12 ng/ml.

FIG. 3 is a graph showing serum concentrations of mouse [D-Leu-4]OB3 5, 10, 20, 40, 60, and 120 min after subcutaneous delivery of 1 mg of peptide to male Swiss Webster mice (n=6 mice per time point). Each value represents mean±SEM. Error bars are contained within the point and ranged between 0.01 and 0.12 ng/ml.

FIG. 4 is a graph showing serum concentrations of mouse [D-Leu-4]OB3 5, 10, 20, 40, 60, and 120 min after intramuscular delivery of 1 mg of peptide to male Swiss Webster mice (n=6 mice per time point). Each value represents mean±SEM. Error bars are contained within the point and ranged between 0.01 and 0.12 ng/ml.

FIG. 5 is a graph showing serum concentration of mouse [D-Leu-4]OB3 5, 10, 20, 40. 60, and 120 min after intranasal delivery of 1 mg of peptide to male Swiss Webster mice (n=6 mice per time point). Each value represents mean±SEM. Error bars are contained within the, point and ranged between 0.01 and 0.12 ng/ml.

### DETAILED DESCRIPTION OF THE INVENTION

Before the present compositions, devices and methods are described, it is to be understood that this invention is not limited to particular compositions, devices, methods, and experimental conditions described, as such compositions, devices, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention may be understood more readily by reference to the following detailed description of specific embodiments and the Examples included therein.

The present invention is based on the discovery that therapeutic compositions comprising of least one drug and at least one surfactant, wherein the surfactant is comprised of at least one alkyl glycoside and/or at least one saccharide alkyl ester are stable, non-toxic, non-irritating, anti-bacterial compositions that increase bioavailability of the drug and have no observable adverse effects when administered to a subject. The ability to administer a peptide demonstrating weight loss activity through direct oral ingestion offers very significant advantages over all other injectable anti-obesity drugs currently under clinical evaluation. These advantages include avoidance of the need for injection, avoidance of needlestick injuries, and avoidance of the cost and inconvenience associated with the safe collection, transport, and disposal of used syringes. Oral absorption also takes place slowly over extended period of time compared to injection, thus extending the period of time in which the active peptide is present in systemic circulation, thus prolonging its action. In contrast, injection typically provides a rapid increase in blood levels and a corresponding rapid elimination dependent largely on the half-life of the peptide in circulation.

Enteral administration of the compositions of the present invention via nasal spray or nasal instillation affords a further unexpected advantage in that a portion of the leptin peptide is absorbed through the nasal mucosa providing a rapid increase in peptide blood levels with a Tmax of approximately 10 minutes, comparable to that achieved by injection. At the same time the bulk of the leptin peptide passes into the GI tract and is absorbed enterally over an extended period of time with a Tmax of approximately 60 minutes. Since eating behavior in overweight individuals is often impulsive, rapid achievement of therapeutic levels of the leptin peptide is believed to be highly desirable. At the same time, sustaining drug levels over a longer period of time increases the time during which the drug remains effective prior to the next meal.

It is shown herein that oral administration of leptin peptides in the absence of at least one of the alkylsaccharides described herein results in little or no increase in blood levels of leptin peptide. Surprisingly, oral administration of leptin peptides in the presence of at least one of the alkylsaccharides described herein is found to result in a very substantial increase in blood levels of the leptin peptide.

Furthermore, it is discovered that intranasal administration of these compositions also results in a very substantial increase in blood levels of leptin peptide with a Tmax similar to that observed for direct oral administration, indicating that a substantial portion of leptin peptide is absorbed enterally. It is further observed, however, that another peak in the serum level of leptin peptide occurs almost immediately following intranasal administration with a Tmax of approximately 10 minutes.

Thus, the absorption profile for compositions of the present invention containing alkylsaccharides and leptin peptides administered intranasally is biphasic in nature with a first peak exhibiting a Tmax of approximately 10 minutes corresponding to pregastric transmucosal absorption. In the examples below, it is determined that certain leptin peptides in combination with certain alkylsaccharides undergo direct oral absorption when administered orally bypassing, bypassing the nasal route of administration, exhibiting a Tmax at approximately 60 minutes.

As used herein, “leptin” encompasses biologically active variants of naturally occurring leptin, as well as biologically active fragments of naturally occurring leptin and variants thereof, and combinations of the preceding. Leptin is the polypeptide product of the ob gene. Example of leptin peptides for use with the present invention are described in Table 1. Typically, the leptin peptide is derived or isolated from a mammal, such as human or mouse. In various embodiments, the leptin peptide is a human leptin including derivatives, fragments, homologs, analogs and variants thereof.

In the absence of alkylsaccharides, little or no, leptin peptide is absorbed through the nasal mucosa. Increasing the concentration of alkylsaccharides results in increased transmucosal absorption through the nasal mucosa.

Since the amount of peptide drug absorbed through the nasal mucosa has been shown to be directly related to the amount of alkylsaccharide present in the formulation administered, it is apparent that the absorption profile for leptin peptides administered intranasally can be modulated in order to increase or decrease the proportion of the leptin peptides that is absorbed through the gastrointestinal tract. The ability to modulate the pharmacokinetic profile to increase onset of action or sustain action over a longer period of time is beneficial in the case of anti-obesity drugs since it provides a means for balancing the effects of rapid satiation of hunger with extension of time during which hunger is satisfied. In a separate aspect of the present invention, it is noted that nasal delivery of peptides often results in direct nose to brain delivery. Many peptide drugs known to affect satiety such as exendin-4, oxyntomodulin and PYY, are known to exert biological effects both on gastrointestinal tract tissues as well as through signaling processes mediated through the brain. Thus, nasal administration as described in the present invention provides the convenience of oral absorption following mucociliary clearance of the drug from the nose with the possibility of improved efficacy through direct nose to brain absorption of these leptin peptides.

A “composition” or “therapeutic composition” as used herein may include an admixture with an aqueous, organic, or inorganic carrier or excipient, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary stabilizing, thickening or coloring agents can be used, for example a stabilizing dry agent such as triulose.

Alkylsaccharides of the invention can be synthesized by known procedures, i.e., chemically, as described, e.g., in Rosevear et al., Biochemistry 19:4108-4115 (1980) or Koeltzow and Urfer, J. Am. Oil Chem. Soc., 61:1651-1655 (1984), U.S. Pat. No. 3,219,656 and U.S. Pat. No. 3,839,318 or enzymatically, as described, e.g., in Li et al., J. Biol. Chem., 266:10723-10726 (1991) or Gopalan et al., J. Biol. Chem. 267:9629-9638 (1992).

Alkylsaccharides of the present invention can include, but are not limited to: alkyl glycosides, such as octyl-, nonyl-, decyl-, undecyl-, dodecyl-, tridecyl-, tetradecyl-, pentadecyl-,hexadecyl-, heptadecyl-, and octadecyl-α- or β-D-maltoside, -glucoside or -sucroside (synthesized according to Koeltzow and Urfer; Anatrace Inc., Maumee, Ohio; Calbiochem, San Diego, Calif.; Fluka Chemie, Switzerland); alkyl thiomaltosides, such as heptyl, octyl, dodecyl-, tridecyl-, and tetradecyl-β-D-thiomaltoside (synthesized according to Defaye, J. and Pederson, C., “Hydrogen Fluoride, Solvent and Reagent for Carbohydrate Conversion Technology” in Carbohydrates as Organic Raw Materials, 247-265 (F. W. Lichtenthaler, ed.) VCH Publishers, New York (1991); Ferenci, T., J. Bacteriol, 144:7-11 (1980)); alkyl thioglucosides, such as heptyl- or octyl 1-thio α- or β-D-glucopyranoside (Anatrace, Inc., Maumee, Ohio; see Saito, S. and Tsuchiya, T. Chem. Pharm. Bull. 33:503-508 (1985)); alkyl thiosucroses (synthesized according to, for example, Binder, T. P. and Robyt, J. F., Carbohydr. Res. 140:9-20 (1985)); alkyl maltotriosides (synthesized according to Koeltzow and Urfer); long chain aliphatic carbonic acid amides of sucrose β-amino-alkyl ethers; (synthesized according to Austrian Patent 382,381 (1987); Chem. Abstr., 108:114719 (1988) and Gruber and Greber pp. 95-116); derivatives of palatinose and isomaltamine linked by amide linkage to an alkyl chain (synthesized according to Kunz, M., “Sucrose-based Hydrophilic Building Blocks as Intermediates for the Synthesis of Surfactants and Polymers” in Carbohydrates as Organic Raw Materials, 127-153); derivatives of isomaltamine linked by urea to an alkyl chain (synthesized according to Kunz); long chain aliphatic carbonic acid ureides of sucrose β-amino-alkyl ethers (synthesized according to Gruber and Greber, pp. 95-116); and long chain aliphatic carbonic acid amides of sucrose β-amino-alkyl ethers (synthesized according to Austrian Patent 382,381 (1987), Chem. Abstr., 108:114719 (1988) and Gruber and Greber, pp. 95-116).

As described above, the hydrophobic alkyl can thus be chosen of any desired size, depending on the hydrophobicity desired and the hydrophilicity of the saccharide moiety. For example, one preferred range of alkyl chains is from about 9 to about 24 carbon atoms. An even more preferred range is from about 9 to about 16 or about 14 carbon atoms. Similarly, some preferred glycosides include maltose, sucrose, and glucose linked by glycosidic linkage to an alkyl chain of 9, 10, 12, 13, 14, 16, 18, 20, 22, or 24 carbon atoms, e.g., nonyl-, decyl-, dodecyl- and tetradecyl sucroside, glucoside, and maltoside, etc. These compositions are nontoxic, since they are degraded to an alcohol and an oligosaccharide, and amphipathic.

As used herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues. The saccharide can be chosen, for example, from any currently commercially available saccharide species or can be synthesized. Some examples of the many possible saccharides to use include glucose, maltose, maltotriose, maltotetraose, sucrose and trehalose. Preferable saccharides include maltose, sucrose and glucose.

The alkylsaccharide of the invention can likewise consist of a sucrose ester. As used herein, “sucrose esters” are sucrose esters of fatty acids. Sucrose esters can take many forms because of the eight hydroxyl groups in sucrose available for reaction and the many fatty acid groups, from acetate on up to larger, more bulky fatty acids that can be reacted with sucrose. This flexibility means that many products and functionalities can be tailored, based on the fatty acid moiety used. Sucrose esters have food and non-food uses, especially as surfactants and emulsifiers, with growing applications in pharmaceuticals, cosmetics, detergents and food additives. They are biodegradable, non-toxic and mild to the skin.

The alkylsaccharides of the invention have a hydrophobic alkyl group linked to a hydrophilic saccharide. The linkage between the hydrophobic alkyl group and the hydrophilic saccharide can include, among other possibilities, a glycosidic, thioglycosidic (Horton), amide (Carbohydrates as Organic Raw Materials, F. W. Lichtenthaler ed., VCH Publishers, New York, 1991), ureide (Austrian Pat. 386,414 (1988); Chem. Abstr. 110:137536p (1989); see Gruber, H. and Greber, G., “Reactive Sucrose Derivatives” in Carbohydrates as Organic Raw Materials, pp. 95-116) or ester linkage (Sugar Esters: Preparation and Application, J. C. Colbert ed., (Noyes Data Corp., New Jersey), (1974)). Further, preferred glycosides can include maltose, sucrose, and glucose linked by glycosidic linkage to an alkyl chain of about 9-16 carbon atoms, e.g., nonyl-, decyl-, undecyl-, dodecyl-, tridecyl-, and tetradecyl sucroside, glucoside, and maltoside. Again, these compositions are amphipathic and nontoxic, because they degrade to an alcohol or fatty acid and an oligosaccharide.

The above examples are illustrative of the types of alkylsaccharides to be used in the methods claimed herein, but the list is not exhaustive. Derivatives of the above compounds which fit the criteria of the claims should also be considered when choosing an alkylsaccharide.

The compositions of the present invention can be administered in a format selected from the group consisting of a tablet, a capsule, a suppository, a drop, a spray, an aerosol, a rapidly dissolvable film or wafer, and a sustained release or delayed burst format. The spray and the aerosol can be achieved through use of an appropriate dispenser. The sustained release format can be erodible microparticulates, swelling mucoadhesive particulates, pH sensitive microparticulates, nanoparticles/latex systems, ion-exchange resins and other polymeric gels. These systems maintain prolonged drug contact with the absorptive surface preventing washout and nonproductive drug loss. The prolonged drug contact is non-toxic to the skin and mucosal surfaces.

The Food and Agriculture Organization (FAO) of the United Nations of the World Health Organization (WHO) has shown that some alkylsaccharides have very high NOAELs, allowing for increased consumption of these alkyl glycosides without any adverse effect. This report can be found on the world wide web at inchem.org/documents/jecfa/jecmono/v10je11.htm. For example, the NOAEL for sucrose dodecanoate, a sucrose ester used in food products, is about 20-30 grams/kilogram/day, e.g. a 70 kilogram person (about 154 lbs.) can consume about 1400-2100 grams (or about 3 to 4.6 pounds) of sucrose dodecanoate per day without any observable adverse effect. Typically, an acceptable daily intake for humans is about 1% of the NOAEL, which translates to about 14-21 grams, or 14 million micrograms to 21 million micrograms, per day, indefinitely. Definitions of NOAELs and other related definitions can be found on the world wide web at epa.gov/OCEPAterms. Similarly, in US 40CFR Part 180.70 (177) 54281-54286, the US EPA cites extensive safety data for alkyl glucosides and as a result has issued a ruling that there is no requirement to establish an upper limit of exposure to these substances. Thus, although some effects may be produced with alkyl glycoside levels anticipated in the present invention, the levels are not considered adverse, or precursors to adverse effects.

Accordingly, a subject treated by either oral or nasal administration with compositions of the invention having at least one alkylsaccharide, e.g. dodecyl maltoside (DDM), at a concentration of about 0.06% to 10% by weight of alkyl glycoside two times per day, or three times per day, or more depending on the treatment regimen would consume approximately 180 micrograms to 30 mg of alkylsaccharide depending upon the dosage, frequency of administration, and route of administration. At the high end of consumption, this would represent approximately 1/500 or 1/1000 of a typically acceptable daily intake level of alkylsaccharides.

Stated another way, alkylsaccharides of the present invention have a high NOAEL, such that the amount or concentration of alkylsaccharides used in the present invention do not cause an adverse effect and can be safely consumed without any adverse effect.

The alkylsaccharide compositions of the invention are typically present at a level of from about 0.01% to 20% by weight. More preferred levels of incorporation are from about 0.01% to 5% by weight, from about 0.01% to 2% by weight, or from about 0.01% to 1%. The alkylsaccharide is preferably formulated to be compatible with other components present in the composition. In liquid, or gel, or capsule, or spray compositions the alkylsaccharide is most preferably formulated such that it promotes, or at least does not degrade, the stability of leptin peptide, or other component in these compositions. Further, the invention optimizes the concentration by keeping the concentration of the alkylsaccharide oral absorption enhancer as low as possible, while still maintaining the desired effect.

The compositions of the invention when administered to the subject, yield enhanced oral delivery of the leptin peptides, with a peak concentration (or Cmax) of the peptides in a tissue, or fluid, or in a blood serum or plasma of the subject that is about 15%, 20%, or 50% or greater as compared to a Cmax of the peptides in a tissue (e.g. CNS), or fluid, or blood plasma or serum following administration in the absence of an alkylsaccharide.

Additionally, the therapeutic compositions of the invention may comprise a pharmaceutically acceptable carrier. A “pharmaceutically acceptable carrier” is an aqueous or non-aqueous agent, for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline, aqueous buffers such as acetate, phosphate, phosphate buffered saline (PBS), citrate, lactate, and the like, or other solvents or vehicles such as glycols, glycerol, and oils such as olive oil, vitamin E, vitamin E succinate, or injectable organic esters. A pharmaceutically acceptable carrier can contain physiologically acceptable compounds that act, for example, to stabilize the active peptides, for example, carbohydrates, such as glucose, sucrose or dextrans, antioxidants, such as ascorbic acid or glutathione, chelating agents, protease inhibitors, low molecular weight proteins or other stabilizers or excipients. A pharmaceutically acceptable carrier can also be selected from substances such as distilled water, benzyl alcohol, lactose, starches, talc, magnesium stearate, polyvinylpyrrolidone, alginic acid, colloidal silica, titanium dioxide, and flavoring agents.

A composition of the invention can be prepared in tablet form by mixing a leptin peptide and one alkylsaccharide according to the invention, and an appropriate pharmaceutical carrier or excipient, for example mannitol, corn starch, polyvinylpyrrolidone or the like, granulating the mixture and finally compressing it in the presence of a pharmaceutical carrier such as corn starch, magnesium stearate or the like. If necessary, the formulation thus prepared may include a sugar-coating or enteric coating or covered in such a way that the active principle is released gradually, for example, in the appropriate pH medium.

The term “enteric coating,” is a polymer encasing, surrounding, or forming a layer, or membrane around the therapeutic composition or core. Also, the enteric coating can contain a drug which is compatible or incompatible with the coating. One tablet composition may include an enteric coating polymer with a compatible drug which dissolves or releases the drug at higher pH levels (e.g., pH greater than 4.0, greater than 4.5, greater than 5.0 or higher) and not at low pH levels (e.g., pH 4 or less); or the reverse.

In an embodiment, the dose dependent release form of the invention is a tablet comprising:

(a) a core comprising:
(i) a leptin peptide; and
(ii) an alkylsaccharide; and
(b) at least one membrane coating surrounding the core, wherein the coating is an impermeable, permeable, semi-permeable or porous coating and becomes more permeable or porous upon contacting an aqueous environment of a defined pH.

The term “membrane” is synonymous with “coating,” or equivalents thereof. The terms are used to identify a region of a medicament, for example, a tablet, that is impermeable, permeable, semi-permeable or porous to an aqueous solution(s) or bodily fluid(s), and/or to the therapeutic agent(s) or drug(s) encapsulated therein. If the membrane is permeable, semi-permeable or porous to the drug, the drug can be released through the openings or pores of the membrane in solution or in vivo. The porous membrane can be manufactured mechanically (e.g., drilling microscopic holes or pores in the membrane layer using a laser), or it can be imparted due to the physiochemical properties of the coating polymer(s). Membrane or coating polymers of the invention are well known in the art, and include cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770, 3,916,899, 4,008,719, 4,036,228 and 4,11210 which are incorporated herein by reference.

Further, the enteric coating according to the invention can include a plasticizer, and a sufficient amount of sodium hydroxide (NaOH) to effect or adjust the pH of the suspension in solution or in vivo. Examples of plasticizers include triethyl citrate, triacetin, tributyl sebecate, or polyethylene glycol. Other alkalizing agents, including potassium hydroxide, calcium carbonate, sodium carboxymethylcellulose, magnesium oxide, and magnesium hydroxide can also be used to effect or adjust the pH of the suspension in solution or in vivo.

Accordingly, in one embodiment, an enteric coating can be designed to release a certain percentage of a drug or drugs in certain mediums with a certain pH or pH range. For example, the therapeutic composition of the invention may include at least one enteric coating encasing or protecting at least one drug which is chemically unstable in an acidic environment (e.g., the stomach). The enteric coating protects the drug from the acidic environment (e.g., pH <3), while releasing the drug in locations which are less acidic, for example, regions of the small and large intestine where the pH is 3, or 4, or 5, or greater. A medicament of this nature will travel from one region of the gastrointestinal tract to the other, for example, it takes about 2 to about 4 hours for a drug to move from the stomach to the small intestine (duodenum, jejunum and ileum). During this passage or transit, the pH changes from about 3 (e.g., stomach) to 4, or 5, or to about a pH of 6 or 7 or greater. Thus, the enteric coating allows the core containing the drug to remain substantially intact, and prevents premature drug release or the acid from penetrating and de-stabilizing the drug.

Examples of suitable enteric polymers include but are not limited to cellulose acetate phthalate, hydroxypropylmethylcellulose phthalate, polyvinylacetate phthalate, methacrylic acid copolymer, shellac, cellulose acetate trimellitate, hydroxypropylmethylcellulose acetate succinate, hydroxypropylmethylcellulose phthalate, cellulose acetate phthalate, cellulose acetate succinate, cellulose acetate malate, cellulose benzoate phthalate, cellulose propionate phthalate, methylcellulose phthalate, carboxymethylethylcellulose, ethylhydroxyethylcellulose phthalate, shellac, styrene-acrylic acid copolymer, methyl acrylate-acrylic acid copolymer, methyl acrylate-methacrylic acid copolymer, butyl acrylate-styrene-acrylic acid copolymer, methacrylic acid-methyl methacrylate copolymer, methacrylic acid-ethyl acrylate copolymer, methyl acrylate-methacrylic acid-octyl acrylate copolymer, vinyl acetate-maleic acid anhydride copolymer, styrene-maleic acid anhydride copolymer, styrene-maleic acid monoester copolymer, vinyl methyl ether-maleic acid anhydride copolymer, ethylene-maleic acid anhydride copolymer, vinyl butyl ether-maleic acid anhydride copolymer, acrylonitrile-methyl acrylate-maleic acid anhydride copolymer, butyl acrylate-styrene-maleic acid anhydride copolymer, polyvinyl alcohol phthalate, polyvinyl acetal phthalate, polyvinyl butylate phthalate and polyvinyl acetoacetal phthalate, or combinations thereof. One skilled in the art will appreciate that other hydrophilic, hydrophobic and enteric coating polymers may be readily employed, singly or in any combination, as all or part of a coating according to the invention.

A composition of the invention in the form of a tablet can have a plurality of coatings, for example, a hydrophilic coating (e.g., hydroxypropylmethyl-cellulose), and/or a hydrophobic coating (e.g., alkylcelluloses), and/or an enteric coating. For example, the tablet core can be encased by a plurality of the same type of coating, or a plurality of different types of coating selected from a hydrophilic, hydrophobic or enteric coating. Hence, it is anticipated that a tablet can be designed having at least one, but can have more than one layer consisting of the same or different coatings dependent on the target tissue or purpose of the drug or drugs. For example the tablet core layer may have a first composition enclosed by a first coating layer (e.g. hydrophilic, hydrophobic, or enteric coating), and a second same or different composition or drug having the same or different dosage can be enclosed in second coating layer, etc. This layering of various coatings provides for a first, second, third, or more gradual or dose dependent release of the same or different drug containing composition.

In one embodiment, a first dosage of a first composition of the invention is contained in a tablet core and with an enteric-coating such that the enteric-coating protects and prevents the composition contained therein from breaking down or being released into the stomach. In another example, the first loading dose of the therapeutic composition is included in the first layer and consists of from about 10% to about 40% of the total amount of the total composition included in the formulation or tablet. In a second loading dose, another percentage of the total dose of the composition is released. The invention contemplates as many time release doses as is necessary in a treatment regimen. Thus, in certain aspects, a single coating or plurality of coating layers is in an amount ranging from about 2% to 6% by weight, preferably about 2% to about 5%, even more preferably from about 2% to about 3% by weight of the coated unit dosage form.

Accordingly, the composition preparations of the invention make it possible for contents of a hard capsule or tablet to be selectively released at a desired site the more distal parts of the gastro-intestinal tract (e.g. small and large intestine) by selecting the a suitable pH-soluble polymer for a specific region. Mechanical expulsion of the composition preparations may also be achieved by inclusion of a water absorbing polymer that expands upon water absorption within a hard semi-permeable capsule thus expelling composition through an opening in the hard capsule.

Further, it will be understood by one skilled in the art, that the specific dose level and frequency of dosage for any particular subject in need of treatment may be varied and will depend upon a variety of factors including the activity of the specific leptin peptide employed, the metabolic stability and length of action of that peptide, the age, body weight, general health, sex, diet, mode and time of administration, rate of excretion, drug combination, the severity of the particular condition, and the subject undergoing therapy.

It has been shown that alkylsaccharides, particularly alkylmaltosides and more specifically, dodecylmaltoside (DDM) and tetradecylmaltoside (TDM), stabilize peptides (U.S. Pat. No. 7,425,542). Thus DDM and TDM are exemplary embodiments of the present invention.

The term “fast-dispersing dosage form” is intended to encompass all the types of dosage forms capable of dissolving, entirely or in part, within the mouth. However, in exemplary aspects, the fast-dispersing dosage form is a solid, fast-dispersing network of the active ingredient and a water-soluble or water-dispersible carrier matrix which is inert towards the active ingredient and excipients. In various embodiments, the network may be obtained by lyophilizing or subliming solvent from a composition in the solid state, which composition comprises the active ingredient, an alkyl saccharide, and a solution of the carrier in a solvent. While a variety of solvents are known in the art as being suitable for this use, one solvent particularly well suited for use with the present invention is water. Water—alcohol mixtures may also be employed where drug solubility in the mixed solvent is enhanced. For poorly water soluble drugs, dispersions of small drug particles can be suspended in an aqueous gel that maintains uniform distribution of the substantially insoluble drug during the lyophilization or subliming process.

In various aspects, the fast-dissolve compositions of the invention disintegrates within 20 seconds, preferably less than 10 seconds, of being placed in the oral cavity. Matrix forming agents suitable for use in fast-dissolve formulations of the present invention are described throughout this application. Such agents include materials derived from animal or vegetable proteins, such as the gelatins, collagens, dextrins and soy, wheat and psyllium seed proteins; gums such as acacia, guar, agar, and xanthan; polysaccharides; alginates; carrageenans; dextrans; carboxymethylcelluloses; pectins; synthetic polymers such as polyvinylpyrrolidone; and polypeptide/protein or polysaccharide complexes such as gelatin-acacia complexes. In exemplary aspects, gelatin, particularly fish gelatin or porcine gelatin is used.

Furthermore, the compositions of the invention can be administered in a format selected from the group consisting of a drop, a spray, an aerosol and a sustained release format. The spray and the aerosol can be achieved through use of the appropriate dispenser. Sustained action agents include gelling agents such as chitosans, microcrystalline cellulose, and pectins or ciliostatic agents such as benzalkonium chloride, metacresol, phenol, resorcinol and the like. These sustained action agents maintain prolonged drug contact with the absorptive surface preventing washout and nonproductive drug loss.

The present invention applies not only to leptin peptide fragments, but also to other small peptides, typically with 15 amino acyl residues or less in size, which may be inherently substantially stable or substantially stabilized upon addition of a suitable protease inhibitor, such as those cited above, when presented to the GI tract.

The present invention is more particularly described in the following examples which are intended as illustrative only since numerous modifications and variations therein will be apparent to those skilled in the art. The following examples are intended to illustrate but not limit the invention.

### Example 1

### Oral Administration of Mouse [D-Leu-4]OB3 Leptin Related Peptide Formulated with n-Dodecyl-β-D-Maltoside (DDM)

This example shows the enteral uptake of anti-obesity peptide mouse [D-Leu-4]OB3 formulated with 0.3% alkylsaccharide (n-dodecyl-beta-D-maltoside) upon oral administration to six-week old male Swiss Webster mice (Taconic Farms, Germantown, N.Y.).

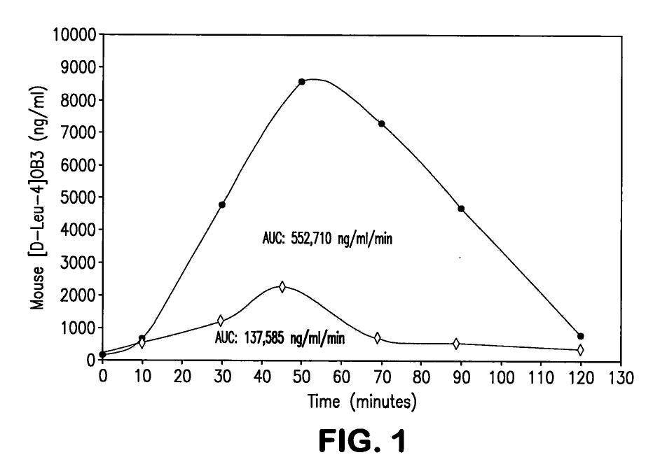

Mouse [D-Leu-4]OB3 (at a concentration of 1 mg/200 μL) was dissolved in either PBS (pH 7.2) or 0.3% n-dodecyl-beta-D-maltoside reconstituted in PBS (pH 7.2) and administered by gavage, without anesthesia, to each of 4 mice per time point. After 10, 30, 50, 70, 90 or 120 minutes, the mice were euthanized by inhalation of isoflurane (5%) and exsanguinated by puncture of the caudal vena cava. Blood was also collected from four mice not given peptide (prebleed). The blood from each of the four mice in the time period was pooled, and serum samples were prepared. Mouse [D-Leu-4]OB3 content of the pooled samples was measured by competitive ELISA. Uptake curves were plotted using Microsoft Excel, and AUC was calculated using a function of the graphics program SigmaPlot 8.0 (SPSS Science, Chicago, Ill.). The lowest AUC value obtained was arbitrarily set at 1.0. Relative bioavailability was determined by comparing all other AUC values to 1.0. The results are shown in FIG. 1 and Table 2 below:

TABLE 2

| | AUC | Relative |
|---|---|---|
| Formulation | (ng/ml/min) | Bioavailability |
| Peptide in PBS | 137,585 | 1.0 |
| Peptide in | 552,710 | 4.0 |
| 0.3% DDM | | |

### Example 2

### Oral Administration of Mouse [D-Leu-4]OB3 Leptin Related Peptide Formulated with n-Tetradecyl-β-D-Maltoside (TDM) and Comparison with Intraperitoneal, Subcutaneous, and Intramuscular Injection

This example shows the enteral uptake of anti-obesity peptide mouse [D-Leu-4]OB3 formulated with 0.3% n-tetradecyl-beta-D-maltoside upon oral administration to six-week old male Swiss Webster mice (Taconic Farms, Germantown, N.Y.).

Doses of 1 mg each were also administered to mice by the IP, SC and IM routes in solutions containing 0.18% tetradecyl beta-D-maltoside and compared to administration by oral gavage in a solution containing 0.3% (3 mg/mL wt/vol) of tetradecyl-beta-D-maltoside.

Six week-old male Swiss Webster mice weighing approximately 30 gm were maintained at a constant temperature (24 C) with lights on from 07:00 to 19:00 h, and allowed food and water ad libitum until used for uptake studies. Mouse [D-Leu-4]OB3 was prepared commercially as a C-terminal amide by Bachem (Torrance, Calif., USA). For sc, im, and ip delivery, the peptide was dissolved in sterile phosphate buffered saline (PBS, pH 7.2) at a concentration of 1 mg/200 μL as described previously containing 0.18% tetradecyl-beta-D-maltoside. For oral gavage [D-Leu-4]OB3 was dissolved in 0.3% dodecyl-beta-D-maltoside reconstituted in PBS (pH 7.2) at a concentration of 1 mg/200 μL. At time zero (0), a single 200 μL sc, im, ip or oral dose of mouse [D-Leu-4]OB3 was given to each of six mice per time point. Following peptide administration, the mice were transferred to separate cages for the designated time period.

Five, 10, 20, 40, 60, or 120 minutes after peptide delivery, the mice were anesthetized with isoflurane (5%) and exsanguinated by cardiac puncture. The blood was collected in sterile nonheparinized plastic centrifuge tubes and allowed to stand at room temperature for 1 h. The clotted blood was rimmed from the walls of the tubes with sterile wooden applicator sticks. Individual serum samples were prepared by centrifugation for 30 min at 2600×g in an Eppendorf™ 5702R, A-4-38 rotor (Eppendorf North America, Westbury, N.Y., USA), and stored frozen until assayed for mouse [D-Leu-4]OB3 content by competitive ELISA.

The [D-Leu-4]OB3 competitive ELISA assay was carried out as follows. 96-well polystyrene plates (Pierce Biotechnology, Inc., Rockford, Ill., USA) were coated with 100 μL of a 5 ug/ml solution of BSA-conjugated mouse [D-Leu-4]OB3 (QED Bioscience, San Diego, Calif.) in carbonate-bicarbonate buffer (pH 9.4). The coated plates were incubated overnight at 4 C. Unoccupied sites were blocked with 200 μL StartingBlock™ in PBS (Pierce Biotechnology Inc., Rockford, Ill., USA) for 2 h at room temperature. Mouse [D-Leu-4]OB3 standards ranging from 5 to 10,000 ng/ml are prepared in PBS (pH 7.2). In a separate incubation, 100 μL of mouse [D-Leu-4]OB3 primary antibody raised in New Zealand White rabbits (QED Bioscience, San Diego, Calif.) and diluted to 1:5000 in StartingBlock™, or 100 μL of the primary antibody+50 μL of each standard or serum sample are added to 500 μL microcentrifuge tubes and incubated for 1 h at 37 C. At the end of the incubation period, 100 μL of each antibody-bound standard or sample were added to the wells and incubated for 1 h at room temperature. HRP-conjugated goat-anti-rabbit IgG (Pierce Biotechnology Inc, Rockford, Ill., USA) was used as the secondary antibody. 100 μL was added to each well and incubated for 1 h at room temperature. At the end of the incubation period, 100 μL of ABTS substrate (Pierce Biotechnology Inc, Rockford, Ill., USA) was added to each well and incubated for 30 minutes on a rotary rocker. Color development was stopped with 1% SDS. Absorbance was read at 405 nm with a Molecular Devices microplate reader (MDS Sciex, Concord, Ontario Canada). Each sample was assayed in triplicate. Intra-assay and inter-assay coefficients of variation are 0.04% and 0.2%, respectively. The relative bioavailability was determined by plotting the serum concentrations of mouse [D-Leu-4]OB3 vs. time following ip, sc, or im delivery using the graphics program SigmaPlot™ 8.0 (SPSS Science, Chicago, Ill., USA). The area under each curve (AUC) was calculated with a function of this program. The relative oral bioavailability compared to the three injection modes is presented below in Table 3. For example, oral gavage achieved 47.3% oral bioavailability compared to subcutaneous injection.

TABLE 3

| | | | Relative Oral |
|---|---|---|---|
| | | | Bioavailability Compared |
| | | AUC | to Three Injection Modes |
| Alkylsaccharide | Delivery | (ng/ | (% of AUC achieved by |
| (%) | Route | ml/min) | each respective injection) |
| 0.3% dodecyl-beta- | Oral | 559,330 | — |
| D-maltoside | gavage | | |
| 0.18% tetradecyl- | IP | 1,072,270 | 52.2% of IP AUC |
| beta-D-maltoside | | | |
| 0.18% tetradecyl- | SC | 1,182,498 | 47.3% of SC AUC |
| beta-D-maltoside | | | |
| 0.18% tetradecyl- | IM | 1,481,060 | 37.8% of IM AUC |
| beta-D-maltoside | | | |

### Example 3

### Enteral Administration of Mouse [D-Leu-4]OB3 via Nasal Instillation

Mouse [D-Leu-4]OB3, a leptin synthetic peptide amide, has shown significant results for the treatment of obesity and type 2 diabetes mellitus in the preclinical setting. To study the pharmacokinetics of mouse [D-Leu-4]0B3 following its administration, this experiment studied the uptake profile, relative bioavailability, serum half-life, clearance, and volume of distribution of mouse [D-Leu-4]OB3 in male Swiss Webster mice following intraperitoneal (ip), subcutaneous (sc), intramuscular (im), and intranasal administration with n-tetradecyl-beta-D-maltoside.

The following materials and methods were used for the study. Six week-old male Swiss Webster mice weighing approximately 30 g were obtained from Taconic Farms (Germantown, N.Y., USA). The animals were housed three per cage in polycarbonate cages fitted with stainless steel wire lids and air filters, and supported on ventilated racks (Thoren Caging Systems, Hazelton, Pa., USA) in the Albany Medical College Animal Resources Facility. The mice were maintained at a constant temperature (24° C.) with lights on from 07:00 to 19:00 h, and allowed food and water ad libitum until used for uptake studies.

Peptide administration was performed as follows. Mouse [D-Leu-4]OB3 was prepared commercially as a C-terminal amide by Bachem (Torrance, Calif., USA). For sc, im and ip delivery, the peptide was dissolved in sterile phosphate buffered saline (PBS, pH 7.2) at a concentration of 1 mg/200 uL, the concentration we have previously shown to be optimum for regulating energy expenditure, glucose levels, and insulin sensitivity in two genetically obese mouse models [13-17]. For intranasal delivery, [D-Leu-4]OB3 was dissolved in 0.18% n-tetradecyl-beta-D-maltoside reconstituted in PBS (pH 7.2) at a concentration of 1 mg/10 uL. At time zero (0), a single 200 ul sc, im, or ip injection of mouse [D-Leu-4] OB3 was given to each of six mice per time point. Intranasal delivery was achieved by lightly anesthetizing the mice with isoflurane (1-4%) and delivering 10 μL of mouse [D-Leu-4]0B3 into the nares using a Gilson® P-20 pipettor. Following peptide administration the mice were transferred to separate cages for the designated time period.

Collection of blood and serum preparation was performed as follows. Five, 10, 20, 40, 60, or 120 min after peptide delivery, the mice were anesthetized with isoflurane (5%) and exsanguinated by cardiac puncture. The blood was collected in sterile nonheparinized plastic centrifuge tubes and allowed to stand at room temperature for 1 h. The clotted blood was rimmed from the walls of the tubes with sterile wooden applicator sticks. Individual serum samples were prepared by centrifugation for 30 min at 2600×g in an Eppendorf™ 5702R, A-4-38 rotor (Eppendorf North America, Westbury, N.Y., USA), and stored frozen until assayed for mouse [D-Leu-4]OB3 content by competitive ELISA. All of these animal procedures were approved by the Albany Medical College Animal Care and Use Committee, and were performed in accordance with relevant guidelines and regulations.

Mouse [D-Leu-4]OB3 competitive ELISA was performed as follows. 96-well polystyrene plates (Pierce Biotechnology, Inc., Rockford, Ill., USA) were coated with 100 μL of a 5 ug/ml solution of BSA-conjugated mouse [D-Leu-4]OB3 (QED Bioscience, San Diego, Calif.) in carbonate-bicarbonate buffer (pH 9.4). The coated plates were incubated overnight at 4° C. Unoccupied sites were blocked with 200 μL StartingBlock™ in PBS (Pierce Biotechnology Inc., Rockford, Ill., USA) for 2 h at room temperature. Mouse [D-Leu-4]OB3 standards ranging from 5 to 10,000 ng/ml were prepared in PBS (pH 7.2). In a separate incubation, 100 μL of mouse [D-leu-4]OB3 primary antibody raised in New Zealand White rabbits (QED Bioscience, San Diego, Calif.) and diluted to 1:5000 in StartingBlock™, or 100 μL of the primary antibody+50 μL of each standard or serum sample were added to 500 μL microcentrifuge tubes and incubated for 1 h at 37° C. At the end of the incubation period, 100 μL of each antibody-bound standard or sample was added to the wells and incubated for 1 h at room temperature. HRP-conjugated goat-anti-rabbit IgG (Pierce Biotechnology Inc. Rockford, Ill., USA) was used as the secondary antibody. 100 μL was added to each well and incubated for 1 h at room temperature. At the end of the incubation period, 100 μL of ABTS substrate (Pierce Biotechnology Inc. Rockford, Ill., USA) was added to each well and incubated for 30 min on a rotary rocker. Color development was stopped with 1% SDS. Absorbance was read at 405 nm with a Molecular Devices microplate reader (MDS Sciex, Concord, Ontario Canada). Each sample was assayed in triplicate. Intra-assay and inter-assay coefficients of variation were 0.04% and 0.2%, respectively.

Several pharmacokinetic parameters were analyzed. Relative bioavailability was determined as follows. Serum concentrations of mouse [D-Leu-4]OB3 vs. time following ip, sc, im, or intranasal delivery were plotted using the graphics program SigmaPlot™ 8.0 (SPSS Science, Chicago, Ill., USA). The area under each curve (AUC) was calculated with a function of this program. The lowest AUC value obtained was arbitrarily set at 1.0. Relative bioavailability was determined by comparing all other AUC values to 1.0.

Serum half-life (t_{1/2}) was determined as follows. The period of time required for the serum concentration of mouse [D-Leu-4)OB3 to be reduced to exactly one-half of the maximum concentration achieved following ip, sc, im and intranasal administration was calculated using the following formula:

t_{1/2}=0.693/kₑₗᵢₘ

kₑₗᵢₘ represents the elimination constant, determined by plotting the natural log of each of the concentration points in the beta phase of the uptake profiles against time. Linear regression analysis of these plots resulted in a straight line, the slope of which correlates to the kₑₗᵢₘ for each delivery method.

Plasma clearance (CL) was determined as follows. Clearance of mouse [D-Leu-4]OB3 from the plasma following ip, sc, im and intranasal delivery was calculated from the AUC using the following equation:

CL=Dose/AUC.

Apparent volume of distribution (V_{d}) was determined as follows. Since the half-life of a drug is inversely related to its clearance from the plasma and directly proportional to its volume of distribution, the apparent volume of distribution of mouse [D-Leu-4]OB3 following ip, sc, im and intranasal delivery was calculated from its half-life and clearance using the following equation:

t_{1/2}=(0.693×V_{d})/CL.

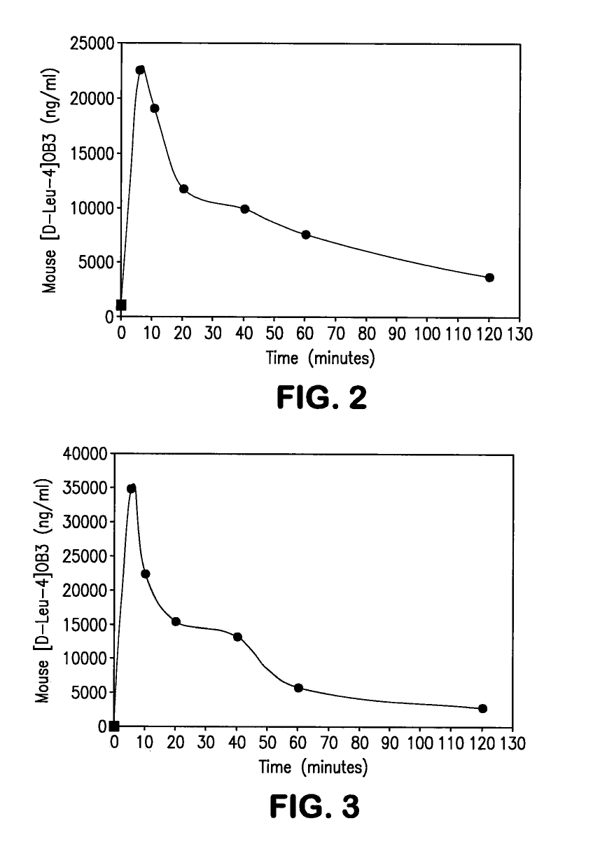

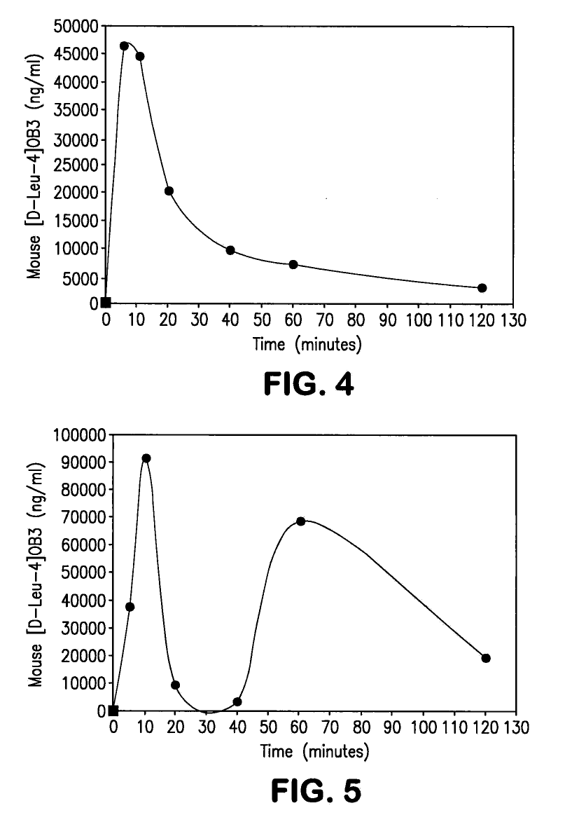

The uptake profiles of mouse [D-Leu-4]OB3 following sc, im, ip, and intranasal delivery are shown in FIGS. 2-5, respectively. Maximum uptake (Cₘₐₓ) of mouse [D-Leu-4]OB3 following ip, sc, and im administration of 1 mg of peptide occurred at 5 min (tₘₐₓ) and gradually decreased with time (FIGS. 2-4). The uptake profile following intranasal administration was conspicuously different from that observed for ip, sc, or im delivery. This profile showed biphasic uptake of mouse [D-Leu-4]OB3 with an initial peak (Cₘₐₓ₁) at 10 min (tₘₐₓ₁) followed by a second peak (Cₘₐₓ₂) of lesser concentration at 60 min (tₘₐₓ₂). Peptide concentrations decreased at different rates after each of the two peaks (FIG. 5). Similar biphasic uptake profiles following intranasal delivery of mouse [D-Leu-4]OB3 formulated with dodecylmaltoside or tetradecylmaltoside in C57BL/6J male mice and male Sprague Dawley rats is also observed.

The relative bioavailability of mouse [D-Leu-4]OB3 was determined by measuring the area under the uptake curve (AUC) for each delivery method. This value represents the total extent of peptide absorption into the systemic circulation, or total uptake, following its administration. The AUC values following ip, sc, and im delivery were 1,072,270 ng/ml/min, 1,182,498 ng/ml/min, and 1,481,060 ng/ml/min, respectively. From these values, the relative bioavailabilities following ip, sc, and im delivery were calculated to be 1.0,1.1, and 1.4 respectively.

Because of the biphasic nature of the uptake profile, the relative bioavailability of mouse [D-Leu-4]OB3 following intranasal delivery was determined by measuring the AUC for each of the two peaks in the profile separately, and determined as follows: AUC=AUC₁+AUC₂. From this value, 4,336,963 ng/ml/min, the relative bioavailability was calculated to be 4.0.

To determine the serum half-life of mouse [D-Leu-4]OB3 following intranasal delivery, the kₑₗᵢₘ for each peak in the curve was calculated separately (kₑₗᵢₘ₁ and kₑₗᵢₘ₂). These values were then used to determine the half-life of mouse [D-Leu-4]OB3 under each peak (t_{1/2 1} and t_{1/2 2}). The overall half-life was calculated as follows: t_{1/2}=t_{1/2 1}+t_{1/2 2}. and determined to be 41.1 min. The serum half-life of mouse [D-Leu-4]OB3 following ip, sc, and im administration was 30.0, 35.0, and 48.8 min, respectively.

The resulting plasma clearance (CL) and apparent volume of distribution (V_{d}) were as follows. Because of the biphasic profile of the uptake curve associated with intranasal delivery of mouse [D-Leu-4]OB3, plasma CL was measured using the AUC associated with each peak in the profile: CL₁=Dose/AUC₁ and CL₂=Dose/AUC₂. Overall clearance was calculated as follows: CL=CL₁+CL₂, and determined to be 1.37 ml/min. Plasma CL following ip, sc, and im administration was 0.93, 0.85 and 0.68 ml/min, respectively.

The apparent volume of distribution of mouse [D-Leu-4]OB3 following intranasal delivery was calculated using the half-life and clearance rate determined for each peak associated with the biphasic uptake profile: t_{1/2 1}=0.693V_{d1}/CL₁, and t_{1/2 2}=0.693×V_{d2}/CL₂. The overall volume of distribution was calculated as follows: V_{d}=V_{d1}+V_{d2} and determined to be 15.4 ml. The apparent volume of distribution following ip, sc, and im delivery was 65.5, 42.9 and 29.4 ml, respectively. All pharmacokinetic parameters measured in this study are summarized in the following Tables 4 and 5.

TABLE 4
Pharmacokinetic parameters of mouse [D-Leu-4)OB3 uptake in male
Swiss Webster mice following i.p., s.c., or i.m. injection of 1 mg
of peptide.

| | Delivery method | | |
|---|---|---|---|
| Parameter | Intraperitoneal | Subcutaneous | Intramuscular |
| Cₘₐₓ (ng/ml) | 22,519 | 35,063 | 46,566 |
| Tₘₐₓ (min) | 5 | 5 | 5 |
| AUC (ng/ml/min) | 1,072,270 | 1,182,498 | 1,481,060 |
| Relative | 1.0 | 1.1 | 1.4 |
| bioavailability | | | |
| kₑₗᵢₘ (ml/min) | 0.0142 | 0.0198 | 0.0231 |
| t_{1/2} (min) | 48.8 | 34.0 | 30.0 |
| CL (ml/min) | 0.93 | 0.85 | 0.68 |
| V_{d} (ml) | 65.5 | 42.9 | 29.4 |

TABLE 5
Pharmacokinetic parameters of mouse [D-Leu-4]OB3 uptake in male
Swiss Webster mice following intranasal delivery of 1 mg of peptide.

| Parameter | Intranasal Delivery Method |
|---|---|
| Cₘₐₓ₁ (ng/ml) | 91,732 |
| tₘₐₓ₁ (min) | 10 |
| Cₘₐₓ₂ (ng/ml) | 69,069 |
| Tₘₐₓ₂ (min) | 60 |
| AUC₁ (ng/ml/min) | 923,953 |
| AUC₂ (ng/ml/min) | 3,413,010 |
| AUC₁ + AUC₂ (ng/ml/min) | 4,336,963 |
| Relative bioavailability | 4.0 |
| kₑₗᵢₘ₁ (ml/min) | 0.0986 |
| kₑₗᵢₘ₂ (ml/min) | 0.0203 |
| t_{1/2 1} (min) | 7.0 |
| t_{1/2 2} (min) | 34.1 |
| t_{1/2 1} + t_{1/2 2} (min) | 41.1 |
| CL₁ (ml/min) | 1.08 |
| CL₂ (ml/min) | 0.29 |
| CL₁ + CL₂ (ml/min) | 1.37 |
| V_{d1} (ml) | 1.1 |
| V_{d2} (ml) | 14.3 |
| V_{d1 +} V_{d2} (ml) | 15.4 |

Continuing interest in the design, development, and preclinical application of leptin peptide agonists suggests that their therapeutic potential in the management of human obesity and its associated metabolic dysfunctions may not be left unexamined much longer. Such small-molecule therapeutics have the potential to be more potent agonists than recombinant leptin, since access to the CNS is not limited by saturable transport across the blood-brain barrier, a suggested locus of leptin resistance. In this regard, it is worthy of special note that demonstration of anti-obesity effects of mouse [D-Leu-4]OB3 in db/db mice indicates a mechanism of action that involves recruitment of signaling pathways that are different from those of leptin. The relevance of this divergence to the management of most cases of human obesity in which leptin resistance results from defective receptor signaling, or in rare cases of genetic obesity resulting from leptin or leptin receptor deficiency, may be great.

Before clinical trials with mouse [D-Leu-4]OB3 can begin, however, questions related to potency, bioavailability, uptake kinetics, half-life, route of administration, and toxicity require attention. The study performed addressed some of these issues, and showed that the uptake kinetics, relative bioavailability, clearance, volume of distribution, and half-life of mouse [D-Leu-4]OB3 are directly related to the route by which the peptide is delivered. The results of this study clearly indicate that intranasal administration of mouse [D-Leu-4]OB3 with tetradecyl-beta-D-maltoside is a more effective method of delivery when compared to sc, im or ip injection methods. This conclusion is supported by the increase in total uptake and enhanced relative bioavailability of mouse [D-Leu-4]OB3 following intranasal delivery which was observed in this study.

While many naturally occurring peptides, such as insulin and growth hormone, have therapeutic application to the treatment of disease, the inherent susceptibility of proteins and peptides to denaturation, proteolytic hydrolysis, and poor absorption from the gastrointestinal tract makes them poor candidates for oral administration. In this regard, reformulation of protein and peptide drugs for administration as a nasal spray or nose drops has the potential for providing a non-invasive and more convenient method of administration, reducing the discomfort and risk of infection associated with injection methods, and fostering higher levels of patient compliance.

Worthy of special note was the biphasic time course associated with the uptake of mouse [D-Leu-4]OB3 following intranasal delivery. This profile showed an initial serum peak which occurred 10 min after administration and a second peak after 60 min. These findings are highly suggestive of a two-compartment model of peptide distribution in which the first peak may be the result of a very rapid systemic absorption through the nasal mucosa, and the second peak representing a much slower absorption of mouse [D-Leu-4]OB3, perhaps from the oropharynx or other parts of the gastrointestinal tract. A two-compartment model following intranasal delivery is further supported by the log scale plot on the y-axis of the serum level decay curve of mouse [D-Leu-4]OB3 vs. time which resulted in a biphasic curvi-linear line (data not shown). In contrast, similar plots of the serum level decay curves associated with ip, sc, or im injection yielded straight lines (data not shown), suggesting a one-compartment model of distribution for each of these methods of peptide delivery.

The enhanced bioavailability of mouse [D-Leu-4]OB3 provided by intranasal delivery with alkyl maltoside when compared to ip, sc, and im injection represents a significant advantage over injection methods of peptide administration. The relevance of this observation to previous studies in ob/ob and db/db mouse models with intraperitoneally administered leptin bioactive peptides is great and suggests that the effects of these peptides on body weight gain, satiety, glycemic control, and insulin sensitivity may be even more pronounced following intranasal administration with alkyl maltoside

Throughout this application, various publications are referenced. The disclosures of these publications in their entireties are hereby incorporated by reference into this application in order to more fully describe the state of the art to which the invention pertains.

### REFERENCES

Zachary M. Novakovic, Matthew C. Leinung, Daniel W. Lee, Patricia Grasso, (2009), Regulatory Peptides, 154:107-111.

Grasso et al. (1997), Endocrinology, 139(4):1413-1418.

U.S. Pat. No. 6,777,388

U.S. Pat. No. 7,186,694

U.S. Pat. No. 7,208,572

Australian Patent 772278

U.S. patent application Ser. No. 11/714,667

U.S. patent application Ser. No. 11/789,762

U.S. Pat. No. 7,425,542

Although the invention has been described with reference to the above examples, it will be understood that modifications and variations are encompassed within the spirit and scope of the invention. Accordingly, the invention is limited only by the following claims.

What is claimed is:

1. A composition comprising:
a) at least one leptin peptide; and
b) at least one alkylsaccharide, wherein the alkylsaccharide provides increased enteral absorption of the leptin peptide.

2. The composition of claim 1, wherein the composition exhibits a biphasic increase in serum levels of the leptin peptide upon nasal administration.

3. The composition of claim 1, wherein the leptin peptide comprises SEQ ID NOS: 1 or 16.

4. The composition of claim 1, wherein the leptin peptide is selected from the group consisting of SEQ ID NOS: 1-16.

5. The composition of claim 4, wherein the peptide is SEQ ID NO: 1 or SEQ ID NO: 16.

6. The composition of claim 5, wherein one to seven amino acids of SEQ ID NO: 1 or SEQ ID NO: 16 is/are substituted with its corresponding D-amino acid isoform.

7. The composition of claim 2, wherein a first Tmax is about 4 to 20 minutes and a second Tmax is about 30 to 120 minutes.

8. The composition of claim 1, wherein the alkylsaccharide has an alkyl chain comprising 10 to 16 carbons.

9. The composition of claim 1, wherein the alkylsaccharide is selected from the group consisting of n-dodecyl-beta-D-maltoside, n-tridecyl-beta-D-maltoside, n-tetradecyl beta-D-maltoside, sucrose mono-dodecanoate, sucrose mono-tridecanoate, and sucrose mono-tetradecanoate.

10. The composition of claim 1, wherein the alkylsaccharide concentration is between about 0.05% and 5%.

11. The composition of claim 10, wherein the alkylsaccharide concentration is between about 0.05% and 0.5%.

12. A method of increasing enteral absorption and systemic circulation of a leptin peptide in a biphasic manner comprising orally or nasally administering to a subject a composition comprising:
a) at least one leptin peptide; and
b) at least one alkylsaccharide, wherein the enteral absorption of the leptin peptide is increased and systemic serum levels of the leptin peptide are increased in a biphasic.

13. The composition of claim 12, wherein the leptin peptide comprises SEQ ID NOS: 1 or 16.

14. The composition of claim 12, wherein the leptin peptide is selected from the group consisting of SEQ ID NOS: 1-16.

15. The composition of claim 14, wherein the peptide is SEQ ID NO: 1 or SEQ ID NO: 16.

16. The composition of claim 15, wherein one to seven amino acids of SEQ ID NO: 1 or SEQ ID NO: 16 is/are substituted with its corresponding D-amino acid isoform.

17. The composition of claim 12, wherein the a first Tmax is about 4 to 20 minutes and a second Tmax is about 30 to 120 minutes.

18. The composition of claim 12, wherein the alkylsaccharide has an alkyl chain comprising 10 to 16 carbons.

19. The composition of claim 12, wherein the alkylsaccharide is selected from the group consisting of n-dodecyl-beta-D-maltoside, n-tridecyl-beta-D-maltoside, n-tetradecyl beta-D-maltoside, sucrose mono-dodecanoate, sucrose mono-tridecanoate, and sucrose mono-tetradecanoate.

20. The composition of claim 12, wherein the alkylsaccharide concentration is between about 0.05% and 5%.

21. The composition of claim 20, wherein the alkylsaccharide concentration is between about 0.05% and 0.5%.

22. The method of claim 12, wherein the Cₘₐₓ is increased greater than 2-fold as compared to delivery without alkylsaccharide.

23. A method of rapidly increasing leptin peptide systemic serum concentration and providing a sustained increase over an extended period of time of leptin peptide systemic serum concentration comprising orally or nasally administering to a subject a composition comprising:
a) at least one leptin peptide; and
b) at least one alkylsaccharide.

24. A method of increasing or decreasing the relative proportion of pregastric absorption of a leptin peptide as compared to gastric absorption comprising:
a) providing a composition, the composition comprising:
i) at least one leptin peptide; and
ii) at least one alkylsaccharide; and
b) increasing or decreasing the concentration of alkylsaccharide;
c) nasally or orally administering the composition to a subject, wherein increasing the concentration of alkylsaccharide increases pregastric absorption.


SEQUENCE LISTING (16 sequences)

SEQ ID NO: 1 - protein, 7 aa, Artificial Sequence
  Synthetic construct
SCSLPQT

SEQ ID NO: 2 - protein, 15 aa, Artificial Sequence
  Synthetic construct
AVPIQKVQDDTKTLI

SEQ ID NO: 3 - protein, 15 aa, Artificial Sequence
  Synthetic construct
TKTLIKTIVTRINDI

SEQ ID NO: 4 - protein, 15 aa, Artificial Sequence
  Synthetic construct
RINDISHTQSVSAKQ

SEQ ID NO: 5 - protein, 15 aa, Artificial Sequence
  Synthetic construct
VSAKQRVTGLDFIPG

SEQ ID NO: 6 - protein, 15 aa, Artificial Sequence
  Synthetic construct
DFIPGLHPILSLSKM

SEQ ID NO: 7 - protein, 15 aa, Artificial Sequence
  Synthetic construct
SLSKMDQTLAVYQQV

SEQ ID NO: 8 - protein, 15 aa, Artificial Sequence
  Synthetic construct
VYQQVLTSLPSQNVL

SEQ ID NO: 9 - protein, 15 aa, Artificial Sequence
  Synthetic construct
SQNVLQIANDLENLR

SEQ ID NO: 10 - protein, 15 aa, Artificial Sequence
  Synthetic construct
DLLHLLAFSKSCSLP

SEQ ID NO: 11 - protein, 15 aa, Artificial Sequence
  Synthetic construct
SCSLPQTSGLQKPES

SEQ ID NO: 12 - protein, 15 aa, Artificial Sequence
  Synthetic construct
QKPESLDGVLEASLY

SEQ ID NO: 13 - protein, 15 aa, Artificial Sequence
  Synthetic construct
EASLYSTEVVALSRL

SEQ ID NO: 14 - protein, 15 aa, Artificial Sequence
  Synthetic construct
ALSRLQGSLQDILQQ

SEQ ID NO: 15 - protein, 12 aa, Artificial Sequence
  Synthetic construct
DILQQLDVSPEC

SEQ ID NO: 16 - protein, 7 aa, Artificial Sequence
  Synthetic construct
SCHLPWA